(19) United States
(12) Patent Application Publication
(10) Pub. No.: US 2005/0004633 A1
(43) Pub. Date: Jan. 6, 2005

(54) Device for applying at least a laser needle on a patient's body

(76) Inventor: Michael Weber, Lauenfoerde (DE)

Correspondence Address: MELVIN K. SILVERMAN, 500 WEST CYPRESS CREEK ROAD, SUITE 500, FT. LAUDERDALE, FL, 33309, US

(21) Appl. No.: 10/490,272

(22) PCT Filed: Sep. 19, 2002
(86) PCT No.: PCT/EP02/10498

(30) Foreign Application Priority Data
Sep. 21, 2001 (DE) 201 15 555.9

(51) Int. Cl. A61B 5/06

(52) U.S. Cl. 607/89

(57) ABSTRACT

The invention concerns a device for applying at least a laser needle on a patient's body. Said device comprises a fixing element capable of being adapted to a part of the patient's body. Said fixing element includes at least a guide for receiving the laser needle so that the latter can be moved relative to the body part by means of said guide.

The invention relates to a device for applying at least one laser needle on a patient's body.

A device for performing acupuncture on a patient by laser radiation is known from DE 200 19 703 U1. A fastening element with an adhesive ring is provided for applying the device on the patient's body.

Applying the laser needle in this way with an adhesive ring is entirely suitable for various types of treatment. However, if, in particular, the body parts concerned are difficult to access, or if the body parts concerned are ones where such an adhesive ring would not have sufficient adhesion, other devices are necessary.

It is therefore an object of the present invention to make available a device for applying at least one laser needle on a patient's body, with which device the at least one laser needle can be applied securely and firmly on the patient's body.

According to the invention, this object is achieved by a fixing element which can be adapted to a body part of the patient and on which there is applied at least one guide arrangement on which the at least one laser needle can be applied in such a way that it can be displaced relative to the body part by means of the guide arrangement.

By means of the fixing element according to the invention and the at least one guide arrangement applied thereon; it is advantageously possible to apply the at least one laser needle on a desired part of the patient's body in such a way that said laser needle is held securely in place yet at the same time is also displaceable.

In this regard, the fixing element adaptable to the body part of the patient guarantees the secure connection of the laser needle to the body part, whereas the at least one guide arrangement provides for the corresponding mobility of the laser needle in relation to the body part.

In an advantageous development of the invention, a plurality of guide arrangements can be applied on the fixing element for application of a plurality of laser needles.

In this way, a very large number of laser needles can be applied on the respective body part of the patient, so that, as is required in traditional acupuncture itself, a plurality of sites on the patient's body can be treated simultaneously.

A particularly stable fixing element is obtained if, in a further advantageous embodiment of the invention, it has a rail adapted to the body part.

To be able to apply this rail on the body part of the patient, it can further be provided that the rail is open on at least one side and can be closed on this side, by means of at least one closure arrangement, in order to form an annular fixing element.

Such a closure arrangement can have a structurally simple design, e.g. a rotatable closure with a plurality of locking positions, or a velcro-type closure, in order to permit straightforward handling thereof.

As an alternative to the closure arrangement, the rail, in a further embodiment of the invention, can also be designed to be resilient so that it clamps onto the body part by means of a spring force. Straightforward handling of the device would be ensured by this means too.

It can further be provided that the rail, on its side facing toward the body part, is provided with a band made of a soft material. This provides protection of the body part on which the rail is applied.

A connection, of the at least one guide arrangement to the fixing element, which is very easy to establish in practice and is very suitable for everyday use can be obtained if the at least one guide arrangement is connected to the fixing element via a sleeve/plug connection.

In this regard, the fixing element can be provided with at least a number of sleeve/plug connections corresponding to the number of laser needles to be applied on the body part. In this way, all the guide arrangements can be made identical and can be interchanged in any desired way.

Each individual laser needle connected to the respective guide arrangement is very easy to orient spatially, and thus in relation to the particular body part of the patient, if the at least one guide arrangement has a flexible guide element connected to the fixing element.

This orientation of the at least one laser needle is simplified if, in a further advantageous embodiment of the invention, a lead element, applied on the flexible guide element and adjustable relative to the latter, is provided with a receiver for the respective laser needle.

If the plug of the sleeve/plug connections is arranged on the glide element, and the sleeves of the sleeve/plug connections are arranged on the fixing element, the guide element can be secured in a particularly simple way on the fixing element.

To be able to maintain a fixed spatial position of the laser needle, and at the same time permits very simple displacement of the latter, it can further be provided that the guide element is designed as a plastically deformable wire.

Protection and, at the same time, stabilization of the plastically deformable wire can be obtained if the wire is covered, along at least approximately its entire length, with a tube.

In this case, a second tube element can be applied on the tube, and the lead element is pushed through said second tube element in order to be able to connect the lead element to the guide element in a particularly simple manner and still guarantee its displaceability.

If the lead element is designed as a rigid wire, it is possible, by displacing the lead element, to position the laser needle applied thereon in a very precise manner with respect to the body part of the patient.

To connect the laser needle to the lead element, a double sleeve, having the receiver for the respective laser needle, can be applied on the lead element.

The device according to the invention can be used to particularly good effect if the body part is the patient's head.

A preferred embodiment of the present invention is set out in principle below.

In the drawing:

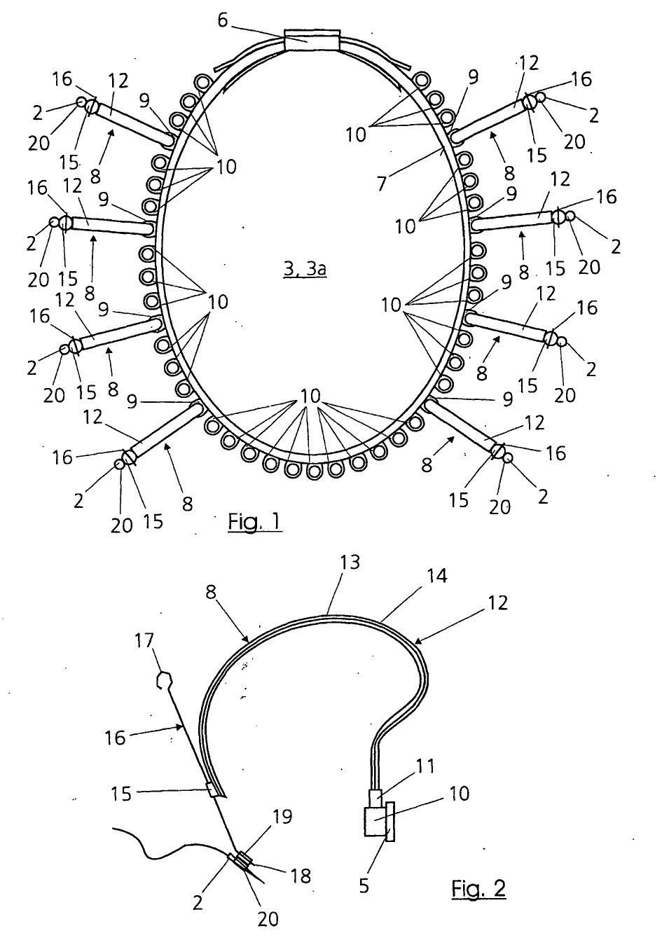

FIG. 1 shows a plan view of a device according to the invention for applying a plurality of laser needles on a patient's body; and

FIG. 2 shows an enlarged representation of one of the guide arrangements from FIG. 1.

FIG. 1 shows a device 1 for applying, in the present case, eight individual laser needles 2 on a body part 3 of a patient, of whom only the body part 3 is represented. The laser needles 2 are intended for performing acupuncture on the patient. In the present case, the body part 3 is the patient's head 3a, to which the device 1 is adapted. It is also possible, however, to apply the device 1 on other body parts, for example on the patient's thigh, or to reconfigure the illustrated device 1 in such a way that they are also suitable for other body parts of the patient. Moreover, it is also possible to apply joust one laser needle 2 or any other desired number of laser needles 2 on the device 1.

The structure and the function of the laser needles 2 are described in detail in DE 200 19 703 U1, for which reason they will not be discussed in detail in the present case. If appropriate, it would also be possible to use the device 1 to apply other laser needles known from the prior art to the body part 3 of the patient.

The device 1 has a fixing element 4 which can be adapted to the particular body part 3 of the patient. For this purpose, the fixing element 4 in the present case has a rail 5 which is adapted to the head 3a and which is open on one side, in the present case in the rear area of the head 3a, and can be closed there by means of a closure arrangement 6. In this way, the rail 5 forms, with the closure arrangement 6, an annular fixing element 4 which, in its closed state, surrounds the entire head 3a. In the opened state of the closure arrangement 6, by contrast, the fixing element 4 can be very easily applied on the head 3a.

In the present case, the closure arrangement 6 is designed as a rotatable closure which has a plurality of locking positions (not shown) in order to be able to adapt the fixing element 4 to different head shapes and sizes. A velcro-type closure or the like, for example, would also be conceivable as a closure arrangement 6. Moreover, it would also be conceivable for the rail 5 to be designed to be resilient so that it clamps onto the body part 3 by means of a spring force and is thus able to function without the closure arrangement 6. In the present case, the rail 5 is made of aluminum, although it is of course also conceivable to make it from another metal or from a suitable plastic.

On its side facing toward the body part 3, the rail 5 is provided with a band 7 which is made of a soft material, for example foam, and thus protects the particular body part 3 from the material of the rail 5 and in this way avoids possible injuries. When the device 1 is used on the head 3a, the band 7 can also additionally provide increased protection against slipping, especially in areas covered with hair.

In an alternative to the design as one circular rail 5, the fixing element 4 could also have a plurality of individual rails 5 which could be held together, for example, by the band 7.

A plurality of guide arrangements 8 for application of in each case one laser needle 2 are applied the fixing element 4. In the present case, the device 1 has a total of eight guide arrangements 8 on each of which one laser needle 2 can be applied, so that the device 1 can be used to apply a total of eight laser needles 2 on the body part 3 of the patient.

Each guide arrangement 8 is connected to the fixing element 4 via a respective sleeve/plug connection 9. In the present case, the fixing element 4 for this purpose has a total of forty-three independent sleeves 10, while each individual guide arrangement 8 has a plug 11 that can be connected to each individual sleeve 10. In a manner not shown, the plugs 11 can of course be secured on the respective sleeve 10 by screws or by other fastening elements. The sleeve/plug connections 9 can, for example, be commercially available lamp-wire terminals.

On the one hand, it would be possible in this way to apply a total of forty-three of the guide arrangements 8 on the fixing element 4. On the other hand, and this is by far the more relevant case in practice, this affords a very large number of possible combinations and variations because the total of eight guide arrangements 8 can be applied at a very wide variety of positions about the circumference of the fixing element 4.

In one embodiment of the device 1 (not shown), it would also be possible to apply the sleeves 10 displaceably on the fixing element 4, so that, it appropriate, only a number of sleeve 10 corresponding to the maximum number of laser needles 2 would have to be applied on the fixing element 4 in order to permit suitable positioning.

As is shown in FIG. 2, each guide arrangement 8 has a flexible guide element 12 which is connected to the fixing element 4 and on which the plug 11 is applied. The guide element 12 in turn has a wire 13 which is made of a plastically deformable material and which is completely covered by a tube 14. In the present case, the wire 13 is made of tin, whereas the tube 14 serving to protect and stabilize the wire 13 is made of silicone. For the wire 13 and the tube 14, it is of course also possible to use other materials which allow the guide element 12 to be brought to a defined position or shape, which it then maintains.

A second, very much shorter tube element 15 is applied on the tube 14, and a lead element 16 is pushed through the opening of said second tube element 15. In the present case, the second tube element 15 is designed in one piece with the tube 14, and in practice it can, for example, be a two-core loudspeaker cable which contains the tube 14 and the tube element 15.

The lead element 16 is designed as a rigid wire which has a handgrip 17 and an end 18 which in the present case is bent at an angle of ca. 20° and on which a double sleeve 19 is applied. The double sleeve 19 is designed like the component made up of tube 14 and tube part 15 and has a tubular receiver 20 for the respective laser needle 2.

When the laser needle 2 has been applied in the receiver 20, and the guide element 12 has been brought into a suitable position, the lead element 16 is used to displace the laser needle 2 if relation to the body part 3 and thus bring it into contact with same. For this purpose, the lead element 16 can be displaced and also turned in the receiver 20.

In this way it is therefore possible to apply up to eight or more laser needles 2 on the body part 3 and bring them into contact with the latter and then carry out a suitable acupuncture treatment, without a large number of persons being needed to hold the laser needles 2.

To give the person operating the device 1 an indication of individual guide arrangements 8, the guide elements 12 and in particular their respective tube 14 can be provided with a specific color.

The diameters of the tube 14, of the tube part 15 and of the double sleeve 19 are configured such that the components to be accommodated in them have a sufficient strength and the least possible mass.

1. A device for applying at least one laser needle on a patient's body, characterized by a fixing element (4) which can be adapted to a body part (3) of the patient and on which there is applied at least one guide arrangement (8) on which the at least one laser needle (2) an be applied in such a way that it can be displaced relative to the body part (3) by means of the guide arrangement (8).

2. The device as claimed in claim 1, characterized in that a plurality of guide arrangements (8) are applied on the fixing element (4) for application of a plurality of laser needles (2).

3. The device as claimed in claim 1 or 2, characterized in that the fixing element (4) has a rail (5) adapted to the body part (3).

4. The device as claimed in claim 4, characterized in that the rail (5) is open on at least one side and can be closed on this side, by means of at least one closure arrangement (6), in order to form an annular fixing element (4).

5. The device as claimed in claim 4, characterized in that the closure arrangement (6) is designed as a rotatable closure which has a plurality of locking positions.

6. The device as claimed in claim 4, characterized in that the closure arrangement (6) is designed as a velcro-type closure.

7. The device as claimed in claim 3, characterized in that the rail (5) is designed to be resilient so that it clamps onto the body art (3) by means of a spring force.

8. The device as claimed in one of claims 3 through 7, characterized in that the rail (5), on its side facing towards the body part (3), is provided with a band (7) made of a soft material.

9. The device as claimed in one of claims 1 through 8, characterized in that the at least one guide arrangement (8) is connected to the fixing element (4) via a sleeve/plug connection (9).

10. The device as claimed in claim 9, characterized in that the fixing element (4) is provided with at least a number of sleeve/plug connections (9) corresponding to the number of laser needles (2) to be applied on the body part (3).

11. The device as claimed in one of claims 1 through 10, characterized it that the at least one guide arrangement (8) has a flexible guide element (12) connected to the fixing element (4).

12. The device as claimed in claim 11, characterized in that a lead element (16), applied on the flexible guide element (12) and adjustable relative to the latter, is provided with a receiver (20) for the respective laser needle (2).

13. The device as claimed in claim 11 or 12, characterized in that the plug (11) of the sleeve/plug connections (9) is arranged on the guide element (12), and the sleeves (10) of the sleeve/plug connections (9) are arranged on the fixing element (4).

14. The device as claimed in one of claims 11, 12 or 13, characterized in that the guide element (12) is designed as a plastically deformable wire (13).

15. The device as claimed in claim 14, characterized in that the wire (13) is covered, along at least approximately its entire length, with a tube (14).

16. The device as claimed in claim 15, characterized in that a second tube element (15) is applied on the tube (14), and the lead element (16) is pushed through said second tube element (15).

17. The device as claimed in one of claims 12 through 16, characterized in that the lead element (16) in designed as a rigid wire.

18. The device as claimed in one of claims 12 through 17, characterized in that a double sleeve (19), having the receiver (20) for the respective laser needle (2), is applied on the lead element (16).

19. The device as claimed in one of claims 1 through 18, characterized in that the body part (3) is the patient's head.
